(19) United States
(12) Patent Application Publication
(10) Pub. No.: US 2008/0131444 A1
(43) Pub. Date: Jun. 5, 2008

(54) Recombiant Protein Expression Systems and Applications Thereof

(75) Inventors: Christophe Brugidou, Pignan (FR); Jean-Loup Lemesre, Montpellier (FR); Florence Piron, Savigny sur Orge (FR); Martine Reyser, Saint-Bauzille De Putois (FR); Christelle Sire, Barcelona (ES)

Correspondence Address: WILMERHALE/BOSTON, 60 STATE STREET, BOSTON, MA, 02109, US

(73) Assignees: Christophe Brugidou; Jean-Loup Lemesre; Florence Piron; Marline Reyser; Christelle Sire; Institut de Recherche Pour le, Paris (FR)

(21) Appl. No.: 11/666,059

(22) PCT Filed: Oct. 21, 2005
(86) PCT No.: PCT/FR05/02626

§ 371 Date: Oct. 22, 2007

(30) Foreign Application Priority Data
Oct. 21, 2004 (FR) 0411212

(51) Int. Cl. A61K 39/12; C12N 15/63; C12N 15/82; A61K 38/00

(52) U.S. Cl. 424/184.1; 435/69.1; 435/320.1; 800/278; 800/294; 514/12

(57) ABSTRACT

The invention relates to a transitory expression system of recombinant proteins in a plant, characterized in that it comprises a viral expression vector comprising a ubiquitous constitutive promoter upstream from a gene of interest or a reporter gene placed downstream from the viral vector or a gene that is adapted for expression of eucaryotic genes or a construction that comprises a gene of interest placed downstream from a constitutive promoter, wherein said constructions are complemented by a construction which also comprises the gene P1 which is placed downstream from the ubiquitous constitutive promoter and which suppresses the silencing of the virus of the yellow variegation of rice (RYMV, Rice Yellow Mottle Virus). Application: production of proteins of therapeutic interest.

The invention relates to the production of recombinant proteins of interest. It relates more particularly to novel systems for expressing such proteins and also to the industrial applications thereof, in particular pharmaceutical applications.

Conventional production systems use the culturing of mammalian cells, insect cells, microorganism cells and cells from transgenic animals. The production costs with these systems are high and do not make it possible to carry out an industrial-scale protein production.

The strategy set up by the inventors is based on the use of viral expression vectors via a bacterium that is pathogenic for the plant.

An objective of the invention is therefore to provide a novel transient expression system for the production of proteins of interest, that makes it possible in particular to reduce production costs and allows a large-scale protein production.

It is also directed toward a novel method for producing a protein using such a transient expression system.

The invention is also directed toward the application of the novel expression system to the production, in particular, of proteins that have vaccinal properties, in particular anti-Leishmania properties.

The system, according to the invention, for the transient expression of proteins in a plant is characterized in that it comprises a viral expression vector comprising an ubiquitous constitutive promoter upstream of a gene of interest or of a reporter gene placed downstream of the viral vector, or of a gene suitable for the expression of eukaryotic genes or of a construct composed of a gene of interest placed downstream of a constitutive promoter, these constructs being complemented by a construct which also comprises, placed downstream of said ubiquitous constitutive promoter, the P1 gene which is a silencing suppressor of the rice yellow mottle virus (RYMV).

Advantageously, the replicative part of RYMV is used upstream of the gene of interest, preferably combined with a silencing suppressor.

In one embodiment of the invention, the system for the transient expression of recombinant proteins is characterized in that said vector is supplemented with one or more independent constructs expressing silencing suppressor genes placed downstream of said ubiquitous constitutive promoter.

The reporter gene is chosen from genes that can be readily detected visually or by fluorimetry, such as the GUS gene which encodes β-glucuronidase.

This GUS gene is placed, in a preferred construct, downstream of the CaMV 35S promoter.

The vector used is in particular a commercial plasmid such as pCambia 1305.1.

These expression systems have many advantages. In particular, they make it possible (i) to produce complex proteins in a eukaryotic system; (ii) to identify the possible level of production, the biological activity of the protein and the production cost; (iii) to identify the best production system (type of plants, of tissues, transient or constitutive systems, constitutive or inducible promoter, and the most suitable protein/suppressor combination); (iv) to work on the biological activity of the protein (mutagenesis, deletion, post-translational modification, targeting sequence for subcellular location).

In accordance with the invention, the method for producing recombinant proteins is characterized in that it comprises the transfer of a vector as defined above into a plant system via a pathogenic bacterium, and the detection of the transient expression of the vector.

Advantageously, the plant systems used are whole plants. As a variant, they are cell suspensions.

In the case of whole plants, plantlets are more particularly used.

Plants which make it possible to obtain satisfactory results include Nicotonia benthamiana, in particular Nicotonia benthamiana var. BY2, and Oryza sativa, in particular Oryza sativa var. O₂₄₂₈.

The transfer into the plant systems is carried out by infiltration of agrobacteria, such as Agrobacterium tumefaciens, or by coculturing.

For the transfer of the vectors into the cell suspensions, bacteria are cocultured with the cell suspensions.

The demonstration of the transient expression is carried out visually, in particular by means of a histochemical test, or quantitatively, in particular by means of a fluorimetric test.

The invention thus provides the means for producing proteins of industrial interest, in particular pharmaceutical interest, in large amounts. In particular, the means of the invention are most particularly suitable for producing proteins with vaccinal properties, for example anti-leishmania properties.

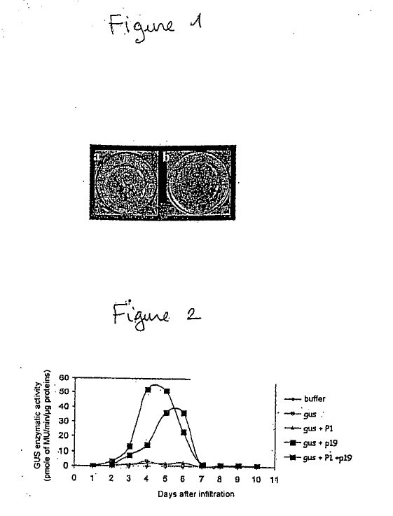

Other characteristics and advantages of the invention will become apparent in the examples which follow and by referring to FIGS. 1 and 2, which represent, respectively:

FIGS. 1A and 1B, the results obtained by means of a histochemical test with Oryza sativa var. O₂₄₂₈ cell suspensions (FIG. 1A) and in BY2 cell suspensions (FIG. 1B), and

FIG. 2, the GUS enzymatic activity in nm of substrate per minute per μg of protein, as a function of time and of expression vectors.

### EXAMPLE 1

### Transit Transfer Of Expression Vectors into the Plant by the Agroinfiltration Method

Agrobacteria are cultured at 28° C. in the presence of the antibiotics essential for the selection of the vector and of the bacteria. After two days of culture, the optical density of the bacteria at 600 nm is estimated using a spectrophotometer. The bacteria are precipitated and resuspended in a solution of MgCl₂ in order to obtain an OD of 0.5. Acetosyringone is added to this solution in order to increase the virulence of the agrobacteria. The solution is incubated at ambient temperature for from 3 to 24 h. The agrobacterial infiltration is carried out with a needleless syringe. A simple pressure exerted on the leaf of the plant allows the solution to infiltrate into the whole leaf. The plants are again placed in a culture chamber and samples are taken at various times after infiltration.

### EXAMPLE 2

### Transfer of Vectors into Cell Suspensions by the Coculture Method

A liquid culture of Agrobacterium tumefaciens is incubated overnight at 28° C. The bacterial culture is plated out on a solid medium and incubated at 28° C. After 2 days, the bacterial layer is resuspended in a liquid medium (medium for culturing BY2 cell suspensions or medium for coculturing Oryza sativa var. O₂₄₂₈). Acetosyringone is added to this medium. The agrobacterial solution is incubated for several hours with the cell suspensions. After washing, the cell suspensions are again placed in the culture chamber.

### EXAMPLE 3

### Demonstration of Transient Expression by Means of a Histochemical Test

The pieces of leaves or cell suspensions sampled are placed in the substrate. After a period of vacuum of 10 min, in order to facilitate penetration of the substrate, the test is placed at 37° C. overnight. A blue coloration of the leaves or of the cells appears when the GUS gene is expressed. FIG. 1 shows the results of the test for the visualization in the Oryza sativa var. O₂₄₂₈ cell suspensions (FIG. 1A) and in the BY2 cell suspensions (FIG. 1B).

The tests on whole plants show that, when infiltration is carried out with agrobacteria containing the GUS reporter gene, a blue coloration of the leaves is observed, revealing expression of the reporter gene.

Enzymatic expression kinetics were performed in order to determine the time for appearance of the protein expression. The results obtained showed that a blue coloration of the leaves appears from 24 h after infiltration onward.

### EXAMPLE 4

### Demonstration of Transient Expression by Means of a Flourimetric Test

The leaves or cells are ground. The proteins are subsequently extracted using a protein extraction buffer (0.5 m Na₂ EDTA, pH 8, N-laurylsarcosine, pure triton, 50 mM NaHPO₄, pH 7) and centrifuged twice. The fluorimetric test is applied to the supernatant. In order to determine the enzymatic activity of the supernatant as a function of the amount of proteins, the proteins are quantified according to the Bradford test. To this effect, Coomassie blue is added to the proteins. After stirring and incubation for 15 min at ambient temperature, the optical density is measured at 595 nm. A standard curve is established with various concentrations of BSA (bovine serum albumin) in order to make the optical density at 595 nm correspond to the amount of protein. A concentration in μg of proteins per μl of samples is then obtained.

The fluorimetric test is based on the hydrolysis of the substrate to a compound that is fluorescent at a wavelength of 460 nm. The samples are added to the substrates. Three measurements of the OD are carried out at 460 nm every 30 min. The difference in OD is calculated and reported per minute, in order to make the optical density at 460 nm correspond to the enzymatic activity, a standard curve is produced with various concentrations of the product derived from the hydrolysis of the substrate. An amount of product is obtained in mole per minute relative to per μg of protein by virtue of the Bradford test quantification. An enzymatic activity is then obtained, in pmole of substrate per minute and per μg of protein.

A quantification of the protein expression by means of the fluorimetric test is carried out.

An enzymatic activity which increases until 3 days after infiltration and which decreases after 6 days is observed.

### EXAMPLE 5

### Use of a Vector Containing a Combination of a Reporter Gene and Silencing Suppressor Genes

In order to increase the protein expression, the vector containing the GUS reporter gene was combined with silencing suppressor genes, P1, p19 or P1 and p19.

When the enzymatic activity kinetics are performed, an expression which is 17 times greater is observed using the p19 silencing suppressor, as shown in FIG. 2.

1. (canceled)

2. The system as claimed in claim 13, further comprising a replicative part of RYMV upstream of the gene of interest.

3. The system as claimed in claim 13, wherein said vector is supplemented with one or more independent constructs expressing silencing suppressor genes placed downstream of said ubiquitous constitutive promoter.

4. The system as claimed in claim 13, wherein the gene of interest is a reporter gene that can be detected visually or by fluorimetry.

5. The system as claimed in claim 13, wherein the GUS gene is placed downstream of the promoter.

6. The system as claimed in claim 13, wherein the vector used is a plasmid.

7. A method for producing recombinant proteins comprising transferring a vector as defined in claim 13 into a plant system via a pathogenic bacterium, and detecting transient expression of the vector.

8. The method as claimed in claim 7, wherein the plant system is a whole plant.

9. The method as claimed in claim 7, wherein the plant system is a cell suspension.

10. The method as claimed in claim 7, wherein the transfer into the plant system is carried out by infiltration of agrobacteria or by coculturing.

11. The method as claimed in claim 9, wherein the transfer of the vector into the cell suspension is carried out by coculturing bacteria with the cell suspension.

12. (canceled)

13. A system for the transient expression of recombinant proteins in a plant, which system comprises a viral expression vector comprising an ubiquitous constitutive promoter
(a) upstream of:
(i) a gene of interest;
(ii) a reporter gene placed downstream of the gene of interest; or
(iii) a gene suitable for the expression of eukaryotic genes; or
(b) downstream of:
(i) a gene of interest;
(ii) a reporter gene placed downstream of the gene of interest; or
(iii) a gene suitable for the expression of eukaryotic genes,
said vector further comprising a P1 gene that is a silencing suppressor of the rice yellow mottle virus (RYMV) downstream of said ubiquitous constitutive promoter.

14. The system as claimed in claim 4, wherein the reporter gene is a GUS gene that encodes β-glucuronidase.

15. The system as claimed in claim 5, wherein the promoter is a CaMV 35S promoter.

16. The system as claimed in claim 13, wherein the promoter is a CaMV 35S promoter.

17. The system as claimed in claim 13, wherein the vector is pCambia 1305.1.

18. A method for producing a protein of interest comprising transferring a vector as defined in claim 13 into a plant system via a pathogenic bacterium, and detecting transient expression of the vector to produce said protein.

19. A method for producing a protein having anti-Leishmania properties comprising transferring a vector as defined in claim 13 into a plant system via a pathogenic bacterium, and detecting transient expression of the vector to produce said protein.

20. A pharmaceutical composition comprising a protein made by the method according to claim 18.

21. A pharmaceutical composition according to claim 20, wherein said composition is a vaccine.

22. The method according to claim 8, wherein the plant is of species Nicotonia benthamiana or Oryza sativa.

23. The method according to claim 22, wherein the plant is Nicotonia Benthamiana var. BY2 or Oryza sativa var. O₂₄₂₈.

24. The method according to claim 10, wherein the agrobacteria is Agrobacterium tumefaciens.

25. The method according to claim 7, wherein transient expression of the vector is detected visually or quantitatively.

26. The method according to claim 25, wherein transient expression of the vector is detected by a histochemical test or a fluorimetric test.
